(19) United States
(12) Patent Application Publication
(10) Pub. No.: US 2022/0401142 A1
(43) Pub. Date: Dec. 22, 2022

(54) PULSE GENERATING CIRCUIT, AND ELECTROSURGICAL GENERATOR INCORPORATING THE SAME

(71) Applicant: CREO MEDICAL LIMITED, Chepstow, Monmouthshire (GB)

(72) Inventors: Christopher Paul HANCOCK, Chepstow (GB); Ilan DAVIES, Chepstow (GB); George HODGKINS, Chepstow (GB)

(21) Appl. No.: 17/781,238

(22) PCT Filed: Nov. 30, 2020
(86) PCT No.: PCT/EP2020/083975

§ 371 Date: May 31, 2022

(30) Foreign Application Priority Data
Dec. 4, 2019 (GB) 1917695.7

(51) Int. Cl. A61B 18/12; H01P 3/06; H03K 3/335

(52) U.S. Cl. CPC A61B 18/1206; H01P 3/06; H03K 3/335; A61B 2018/126

(57) ABSTRACT

A bipolar pulse generating circuit for an electrosurgical generator generates a waveform for electroporation of biological tissue comprising a voltage source connectable to a load via a switching element, and a coaxial transmission line having an inner conductor separated from an outer conductor. The inner conductor first end is connected between the switching element and the voltage source and second end is in an open circuit condition, whereby the line is charged when the switching element is OFF and discharged when the element is ON. The bipolar pulse generating circuit has an output connectable to the load, wherein the first output supports a positive pulse when the line discharges, and a second output supports a negative pulse when the line discharges. The impedance of the coaxial transmission line matches a sum of impedance of the switching element, the load at the first output, and the load at the second output.

### TECHNICAL FIELD

The present invention relates to an electrosurgical generator for generating a waveform suitable for causing electroporation of biological tissue. In particular, the invention relates to a pulse generating circuit for an electrosurgical generator, where the pulse generating circuit is configured to generate high voltage pulses having a duration less than 10 ns.

### BACKGROUND TO THE INVENTION

Electrosurgical generators are pervasive throughout hospital operating theatres, for use in open and laparoscopic procedures, and are also increasingly present in endoscopy suites. In endoscopic procedures the electrosurgical accessory is typically inserted through a lumen inside an endoscope. Considered against the equivalent access channel for laparoscopic surgery, such a lumen is comparatively narrow in bore and greater in length.

WO 2019/185331 A1 discloses an electrosurgical generator capable of supplying energy in a waveform that causes electroporation in biological tissue. The electrosurgical generator may comprise an electroporation waveform supply unit that is integrated with means for generating microwave electromagnetic signals and radiofrequency electromagnetic signals for treatment. The electrosurgical generator may be configured to deliver different types of energy along a common feed cable. The electroporation waveform supply unit comprises a DC power supply and a DC pulse generator. The DC power supply may include a DC-DC converter for up-converting a voltage output by an adjustable voltage supply. Each DC pulse may have a duration in the range 1 ns to 10 ms and a maximum amplitude in the range 10 V to 10 kV.

In recent years there have been numerous developments of ultrashort electric field pulse generators [1]. Ultrashort electric field pulses in the nanosecond regime have numerous applications. Applications includes: measurement of particles, photography, ultra-wideband radar detection and medical application to name a few [2]-[3].

There are numerous methods of generating high amplitude, nanosecond pulsed electric field with a rise and fall time of 2 ns. Traditionally, coaxial transmission line-based implementations, such as Blumlein, in correlation with spark-gap, Marx bank, or diode and laser opening switch techniques have been used to generate high-voltage nanosecond pulses [1].

### SUMMARY OF THE INVENTION

At its most general the present invention provides a pulse generating circuit for an electrosurgical generator, which is configured to generate high voltage pulses having a duration less than 10 ns suitable for causing electroporation of biological cells. In particular, the pulse generating circuit disclosed herein may be suitable for generating bipolar pulses that exhibit a ‘flat-top’ profile, i.e. having steep (e.g. less than 2 ns) rise and fall times, with minimal ringing. As explained in more detail below, this can be achieved through an open circuit transmission line technique in conjunction with placing a pair of load outputs (for positive and negative pulses) in suitable locations relative to the transmission line and a fast switching element.

According to the invention there is provided a bipolar pulse generating circuit for an electrosurgical generator, the bipolar pulse generating circuit comprising: a voltage source connectable to a load via a switching element; a coaxial transmission line having an inner conductor separated from an outer conductor by a dielectric material, wherein the inner conductor has a first end connected between an input of the switching element and the voltage source and a second end in an open circuit condition, whereby the coaxial transmission line is charged by the voltage source when the switching element is in an OFF state and to be discharged when the switching element is in an ON state; a first output connectable to the load, wherein the first output is located between an output of the switching element and ground to support a positive pulse when the coaxial transmission line discharges; and a second output connectable to the load, wherein the second output is located between the outer conductor of the coaxial transmission line and ground to support a negative pulse when the coaxial transmission line discharges, wherein the impedance of the coaxial transmission line is configured to match a sum of (i) the impedance the switching element, (ii) the impedance of the load at the first output, and (iii) the impedance of the load at the second output. With this structure, the circuit can generate a bipolar pulse of ultrashort (e.g. less than 10 ns) duration in which the positive and negative parts of the pulse are symmetrical. Matching the impedances ensures that reflection is minimised or eliminated. This circuit configuration can yield a flat top pulse (due to the matched impedance condition) having a short duration (controlled by the length of the coaxial transmission line).

In one example, a delay line may be connected to either the first output or the second output, whereby supply of the positive pulse and negative pulse at the first output and second output occurs sequentially. For example, a delay line may be connected between the outer conductor of the coaxial transmission line and the second output. The length of the delay line is selectable (or adjustable) to control the amount by which the negative pulse appears at the second output relative to the beginning of the positive pulse. It is possible to include a delay line connected at each of the first output and second output in order to provide full control of the profile of the output bipolar waveform. In particular, it may be desirable to provide a delay line having an adjustable length, so that the separation of the positive and negative pulses can be independently controlled. The delay line may have any suitable structure. For example, it may be another length of coaxial transmission line.

The switching element may comprise: a plurality of series connected avalanche transistors; and a trigger pulse generator configured to generate a trigger pulse to activate the plurality of series connected avalanche transistors. This enables the positive and negative pulses to have ultrashort rise and fall times and an amplitude suitable for electroporation due to the cascading effect of the series connected avalanche transistors. In particular, the amplitude of the output may be 500 V or more, e.g. 1 kV or more, without exceeding the collector-base breakdown voltage across any of the plurality of series connected avalanche transistors.

The coaxial transmission line may have a length selected to provide a line delay equal to or less than 5 ns. The pulse duration is twice the line delay, so the output pulse may have duration equal to or less than 10 ns.

The coaxial transmission line may be charged by the voltage source through a resistor having a high impedance, e.g. 1 MΩ. The circuit may thus be considered as comprises a first loop when the switching element is in an OFF state and a second loop when the switching element is in an ON state. In the first loop, current flows from the voltage source through the resistor to charge the coaxial transmission line. In the second loop, current flows from the coaxial transmission line through the switching element to the load.

The trigger pulse may comprise a TTL signal. The trigger pulse generator may be any source suitable for generating such a signal, e.g. a microprocessor or the like. The trigger pulse may have a voltage less than the emitter-base breakdown voltage of each of the plurality of avalanche transistors.

The duration of the trigger pulse may be longer than the duration of the pulse from the coaxial transmission line, to ensure that the switching element is in an ON state for long enough for the coaxial transmission line to completely discharge. In one example, the trigger pulse has a voltage of 5 V and a duration of 600 ns.

The trigger pulse generator may be connected to the plurality of series connected avalanche transistors via a transformer. This means that the trigger signal is floating between the base and emitter, and is therefore independent of the voltage through the transistor and on to the load. In one example, the trigger pulse may be applied between the collector and emitter of a first transistor of the plurality of series connected avalanche transistors. The first transistor may be the transistor that is furthest from the coaxial transmission line.

A diode may be connected in parallel with each of the plurality of series connected avalanche transistors to clamp the voltage across each transistor to less than its collector-base breakdown voltage. This protects the transistors.

Each transistor in the plurality of series connected avalanche transistors may be identical so that a voltage of the voltage source is divided evenly between the transistors.

As mentioned above, the present invention is particularly suited for use in electrosurgery. The load may therefore comprise an electrosurgical instrument capable of delivered a monopolar pulse for electroporation of biological tissue.

In another example, the invention may provide an electrosurgical generator having a pulse generating circuit as set out above. The pulse generating circuit may be configured to generate an electroporation waveform, i.e. a burst of energy suitable for causing electroporation of biological tissue. The electroporation waveform may comprise one or more rapid high voltage pulses. Each pulse may have a pulse width in a range from 1 ns to 10 μs, preferably in the range from 1 ns to 10 ns, although the invention need not be limited to this range. Shorter duration pulses (e.g. equal to or less than 10 ns) may be preferred for reversible electroporation.

Preferably the rise time of each pulse is equal to or less than 90% of the pulse duration, more preferably equal to or less than 50% of the pulse duration, and most preferably equal to or less than 10% of the pulse duration.

Each pulse may have an amplitude in the range 10 V to 10 kV, preferably in the range 1 kV to 10 kV. Each pulse may be positive pulse from a ground potential.

The electroporation waveform may be a single pulse or a plurality of pulses, e.g. a period train of pulses. The waveform may have a duty cycle equal to or less than 50%, e.g. in the range 0.5% to 50%.

### BRIEF DESCRIPTION OF THE DRAWINGS

Embodiments of the invention are discussed below with reference to the accompanying drawings, in which:

FIG. 1 is a schematic diameter that illustrates the principle of a discharge line generator with an ideal switch;

FIG. 2A is a graph showing a voltage waveform at (i) the transmission line, and (ii) the load in FIG. 1;

FIG. 3A is a schematic diagram representing the open circuit transmission line of FIG. 1 in a DC model;

FIG. 3B is a schematic diagram representing the open circuit transmission line of FIG. 1 in a transmission line model;

FIG. 4 is a schematic diagram of showing the open circuit transmission line of FIG. 1 with an avalanche transistor to generate a positive ultrashort electric field pulse;

FIG. 5 is a diagram of a simulated LTSpice circuit of a monopolar ultrashort electric field pulse generator;

FIG. 6 is a graph showing pulses of various durations generated from the LTSpice circuit of FIG. 5;

FIG. 7 is a monopolar positive pulse observed with a matched 35Ω load, from circuit in FIG. 5;

FIG. 8 is a schematic diagram of showing the open circuit transmission line of FIG. 1 with an avalanche transistor to generate a negative ultrashort electric field pulse;

FIG. 9 is a diagram of a simulated LTSpice circuit of a monopolar ultrashort electric field pulse generator configured to generate a negative pulse;

FIG. 10 is a monopolar negative pulse observed with a matched 35Ω load, from circuit in FIG. 9;

FIG. 11A is a diagram of a simulated LTSpice circuit of a bipolar ultrashort electric field pulse generator without delay lines;

FIG. 11B is a diagram of a simulated LTSpice circuit of a bipolar ultrashort electric field pulse generator with delay lines before the load;

FIG. 12A is a graph depicting voltage observed at various points in the circuit of FIG. 11A; and

FIG. 12B is a graph depicting voltage observed at various points in the circuit of FIG. 11A.

### DETAILED DESCRIPTION, FURTHER OPTIONS AND PREFERENCES

Generation of ultra-short pulses is possible by using an open circuit coaxial transmission line as a high-Q storage element consisting of distributed series of inductors and shunt capacitors with minimal resistance and shunt conductance. Discharging an open ended delay line via a fast switching element provides a means of producing a ‘flat-top’ rectangular pulse with steep fall times of less than 2 ns in a simple and affordable manner. The co-axial transmission line with a characteristic impedance, Z_{c}, a length of l, and a dielectric constant ε_{y}, is charged to a voltage level, V_{cc}, through a high impedance resistor, R_{c}. The line will have and associated delay time T given by the following equation:

$$T = \frac{l \sqrt{{ε}_{r}}}{c}$$

where c is the speed of light (2.99×10⁸ m/s).

It follows from this that the pulse duration associated with the transmission line is:

$$2 T = 2 \frac{l \sqrt{{ε}_{r}}}{c}$$

An ultrashort electric field pulse can be generated on a load, R_{L}, by discharging the transmission line through R_{L} by closing a switching element. The switching element determines the rise time of the ultrashort electric field pulse whilst the transmission line determines the pulse duration (or width) and the fall time.

As explained above, the duration of the pulse at the load will be twice the associated delay time of the transmission line.

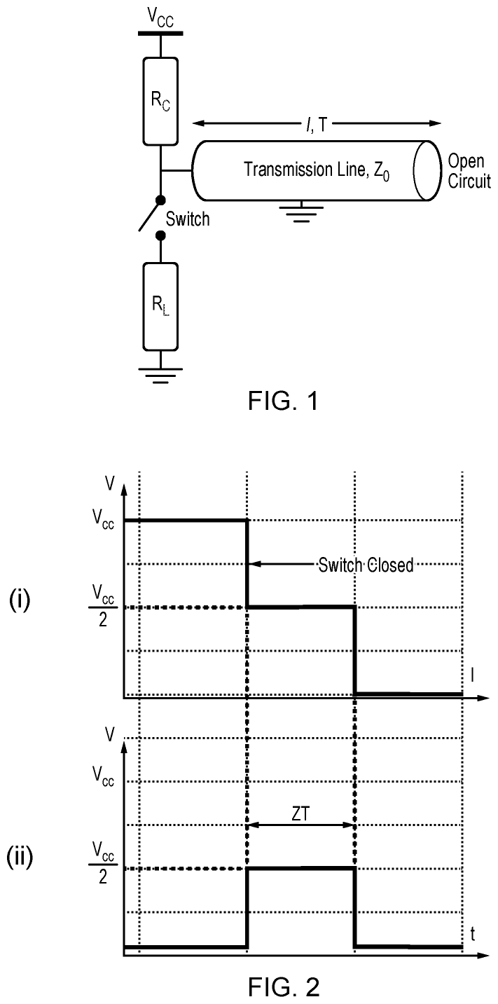

FIG. 1 illustrates the principle of an open circuit transmission line technique with an ideal switch as the switching element.

FIG. 2 shows the voltage waveforms obtained from the system of FIG. 1 at (i) the transmission line Z_{c} and (ii) load R_{L}.

The relationship between the characteristic impedance of the transmission line Z_{c} and the load R_{L} is integral to the performance of an open circuit coaxial transmission line technique in two ways, which can be understood by modelling the configuration using direct circuit (DC) theory and transmission line theory.

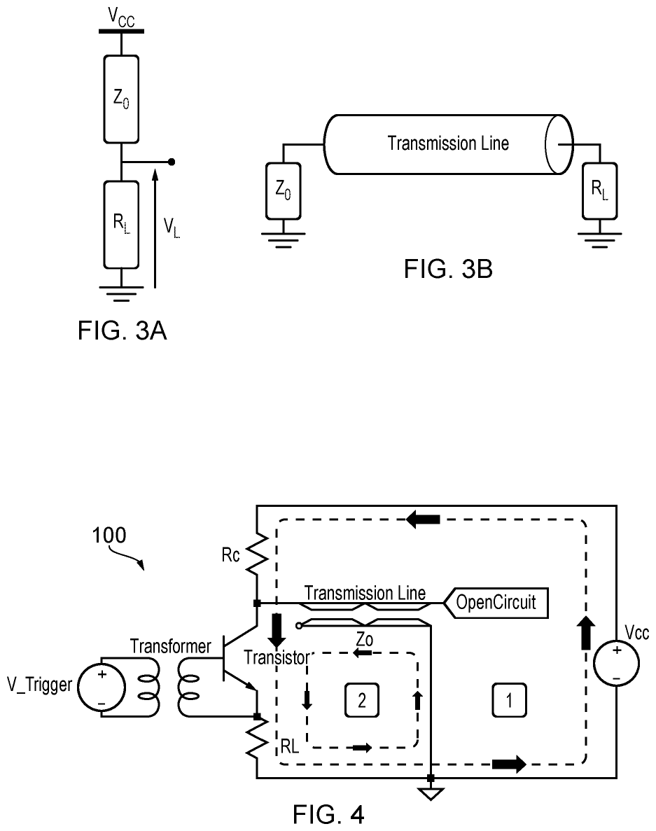

In DC theory, the relationship between Z₀ and R_{L} imitates a potential divider, as shown in FIG. 3A. Their relationship determines the pulse amplitude at the load V_{L}:

$${V}_{L} = ( \frac{{R}_{L}}{{R}_{L} + {Z}_{0}} ) {V}_{cc}$$

If the impedance Zᵤ is the same as R_{L}, the maximum amplitude of the pulse at the load, V_{Lmax}, will be half the voltage the to which the transmission line is charged:

$$if {R}_{L} = {Z}_{0} , {V}_{Lmax} = \frac{{V}_{cc}}{2}$$

Using a transmission line model, the system can be represented as shown in FIG. 3B. In this model, the relationship between Z₀ and R_{L} determines the reflection coefficient, and therefore the pulse shape at the load. If R_{L} is the same as Z₀, the reflection coefficient will be zero and no secondary pulse or reflection of the primary pulse will be seen at the load:

$$Γ = ( \frac{{R}_{L} - {Z}_{0}}{{R}_{L} + {Z}_{0}} ) ∴ if {R}_{L} = {Z}_{0} , Γ = 0$$

Thus, the relationship of Z₀ and R_{L} determine two key aspects of the pulse at a load: (i) the pulse amplitude, and (ii) pulse shape (caused by any reflection). It follows from the analysis above, that the best pulse shape and parameters, the characteristic impedance of the transmission line Z₀ and the load R_{L} should match.

Other features of the pulse are controlled by other parameters of the circuit. For example, the pulse risetime is determined by the behavioural of the switching element, whilst the pulse width is determined by the length of the transmission line, as discussed above.

This switching element in embodiments of the invention is preferably provided by a stacked array of avalanche transistors. An avalanche transistor is known to provide reliable and repeatable high-speed switching of high voltages with rise times as low as 300 μs, which can be achieved in practice if microwave component layout techniques are considered when the circuit are implemented. Avalanche transistors utilize the negative-resistance characteristics region of bipolar junction transistors, which result from operation in the common-emitter breakdown region. The avalanche region lies between collector emitter (V_{CEO}) and collector base (V_{CBO}) voltage when the base current I_{B}=0 A and emitter current I_{E}=0 A.

FIG. 4 is a schematic diagram of a pulse generating circuit 100 that utilises an open circuit transmission line technique in combination with an avalanche transistor as a fast switching element. The circuit function is based on the discharge of the open-circuit transmission line across an avalanche transistor into a load R_{L}.

A single avalanche transistor circuit can be configured to have a bi-stable operation, where the maximum pulse amplitude at the output is limited to half the value of the transistor's collector-emitter breakdown voltage, BV_{CES}, if Z₀=R_{L}. A supply voltage V₀ above the transistor's BV_{CES} would permanently breakdown and damage the avalanche transistors as a switching element.

Initially, energy is stored in a co-axial transmission line via a small current flow in loop 1. A positive trigger on the base of the transistor will suddenly switch the transistor ‘on’. The energy stored in the transmission line will simultaneously be released as a high current along loop 2, producing a pulse on R_{L}. The width of the trigger on the base is longer than 2T, i.e. the required pulse width at the load.

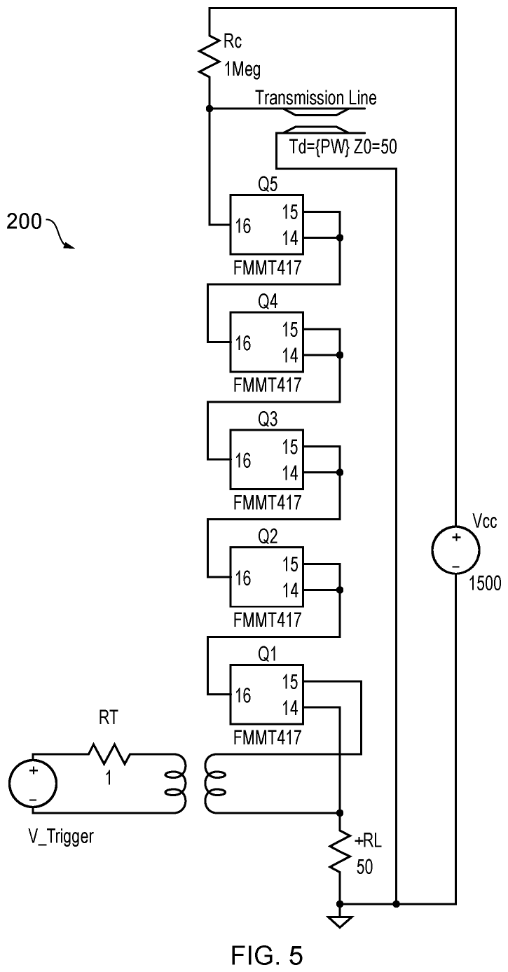

FIG. 5 shows a pulse generation circuit 200 that is an embodiment of the invention. The pulse generation circuit 200 is similar to the circuit shown in FIG. 4, except that in place of the single avalanche transistor, there is a plurality (five in this example) of series-connected avalanche transistors. The plurality of series-connected avalanche transistors effectively operate in combination as a single avalanche transistor. This means that the discharge of the open-circuit transmission line is across the stacked transistors to the load, thereby resulting in a cascade effect that causes a proportionally higher pulse amplitude at the load. In this example, each of the avalanche transistors is identical so that the supply voltage V_{cc} is equally distributed across each of the avalanche transistor in the series chain.

In this arrangement, the maximum pulse amplitude that can be generated is dependent on the number of stacked avalanche transistor n. The number of avalanche transistors required to generate a specific pulse amplitude V_{L} can be expressed as

$${V}_{L} = {nBV}_{CBO} ( \frac{{R}_{L}}{{R}_{L} + {Z}_{0}} )$$

where BV_{CB0} is the collector-base breakdown voltage of each avalanche transistor. If R_{L}=Z₀, a maximum pulse amplitude V_{Lmax} can thus be expressed as

$${V}_{Lmax} = \frac{{nBV}_{CBO}}{2}$$

In the pulse generating circuit 200 five FMMT417 avalanche transistor are stacked. Each transistor has an collector-emitter breakdown voltage BV_{CEO} of 100 V and a collector-base breakdown voltage BV_{CEO} of 320 V. The circuit shown in FIG. 5 was simulated using LTSpice models. The Spice model of the FMMT417 was directly taken from the manufacture's website. The source resistance R_{c} is 1 MA, characteristic impedance of the transmission line Z₀ is 50Ω, source voltage V_{cc} is 1.5 kV.

The circuit may include a diode (not shown) connected in parallel with each transistor to clamp the voltage to ensure that the voltage across each transistor does not exceed its collector-base breakdown voltage. Doing so can increase the lifespan of the transistors and ensure that triggering occurs by the trigger signal.

The trigger signal may be provided by any suitable source. Preferably the trigger signal is generated by a TTL source or a microcontroller. In this example, the trigger signal comprises a pulse having a duration of 600 ns and a 5 V amplitude and pulse period (period of repetition) of 20 ms. It is advantageous to have a 5 V trigger signal because it is less than the emitter-base breakdown voltage of the transistors.

The pulse width of trigger signal is arranged to be longer than the pulse desired to be generated from the transmission line. The duration of 600 ns was chosen in this case to provide a safe margin to allow the whole transmission line to discharge.

The trigger signal repetition rate (pulse period) is limited by the time it takes for the open-circuit charged transmission line to charge up again to full capacity.

A transformer is disposed between the trigger signal generator and the base and emitter of the first transistor in the stack (i.e. the transistor furthest from the transmission line). This configuration means that the trigger pulse is floating, and therefore should be the same between the base and emitter of the first transistor no matter the voltage through the transistor and onto the load. As a result, the amplitude of the pulse at the load ought to increase linearly with the number of transistors in the stack. The transformer may be a 1-EMR-046 Gate Drive Transformer having a 1:1 winding ratio and high voltage isolation.

In use, the five stacked avalanche transistors are initially in their off-state, with each transistor having 300 V across them (i.e. V_{cc}/n). When a positive trigger signal is applied to the base of the first transistor Q1, Q1 is turned ‘on’ and places its collector voltage near ground potential. This results in the second transistor Q2 having twice the collector-emitter voltage, thus creating the desired condition in terms of overvolting and therefore causes a non-destructive avalanching of Q2 and places its collector near ground potential. This creates a sequential ‘knock-on’ effect on the next transistor in the chain resulting in the overvolting of the first avalanche transistors, Q1, to the final avalanche transistors, Q5 near the charged open circuit transmission line. When Q5 is turned ‘on’, a fast rise time is produced at the load (<2 ns), therefore allowing the charged open circuit transmission line to discharge through the load producing a pulse with a width of 2T and a maximum amplitude of V_{cc}/2, if R_{L}=Z₀.

The pulse generating circuit 200 may thus be used to generate monopolar ultrashort electric field pulses.

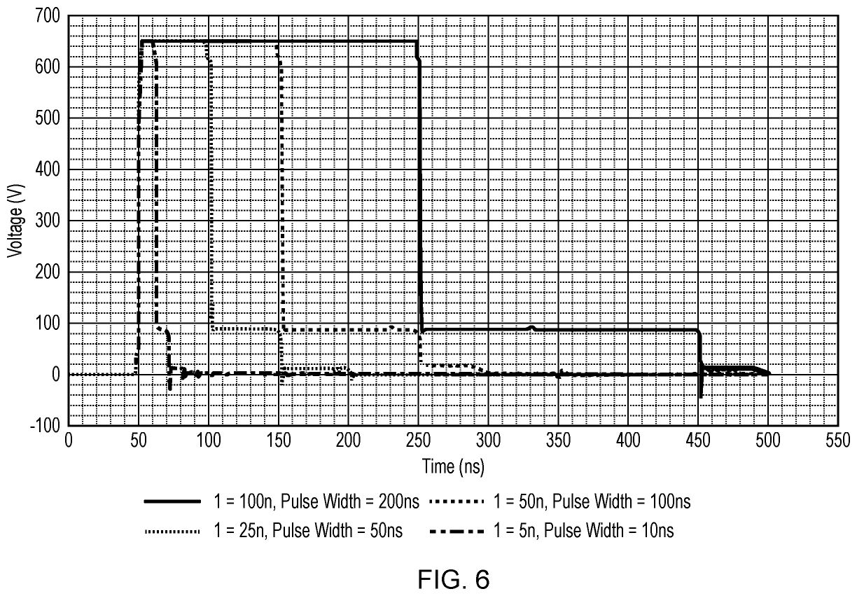

FIG. 6 is a graph showing voltage pulses obtained for a range of transmission line lengths. In FIG. 6, the transmission line lengths are characterised by the line delay T. The graph demonstrate that the transmission line length determines the pulse width of 2T, i.e. transmission lines having line delays of 5 ns, 25 ns, 50 ns and 100 ns produce pulse widths of 10 ns, 50 ns, 100 ns and 200 ns respectively. Additionally, the rise times of all four pulses are the same and less than 2 ns, which emphasises that the switching element, i.e. the five avalanche transistors, determines this factor.

The graph in FIG. 6 suggests that a 50Ω load does not match the transmission line characteristic impedance because secondary pulse of lower amplitude to the primary pulse is seen on each signal. This suggested an unmatched load due to reflection, i.e. Γ≠0. The inventors have realised that it is necessary to compensate for the impedance of the transistors in order to optimise the pulse generation circuit. In the example shown in FIG. 5, each individual transistor has an impedance of ˜3Ω. Therefore, a total of ˜15Ω is across the transistor stack. The reflection coefficient can thus be expressed as

$$Γ = ( \frac{{R}_{Σ} - {Z}_{0}}{{R}_{Σ} + {Z}_{0}} ) = ( \frac{( {R}_{L} + {nR}_{A} ) - {Z}_{0}}{( {R}_{L} + {nR}_{A} ) + {Z}_{0}} )$$

wherein the R_{z} is the total impedance of the circuit, and R_{A} is the impedance of a signal avalanche transistor.

This explains the reflection observed in the pulses shown in FIG. 6, as Γ=0.13, and the amplitude of the reflection pulse is ˜13% of the primary pulse (R_{L}=50Ω, nR_{A}=(3Ω×5)=15Ω and Zᵤ=50Ω). The additional impedance of nR_{A} also affects the DC component of the design, which can be rewritten as:

$${V}_{L} = ( \frac{{R}_{L}}{{Z}_{0} + {R}_{A} + {R}_{L}} ) {V}_{cc}$$

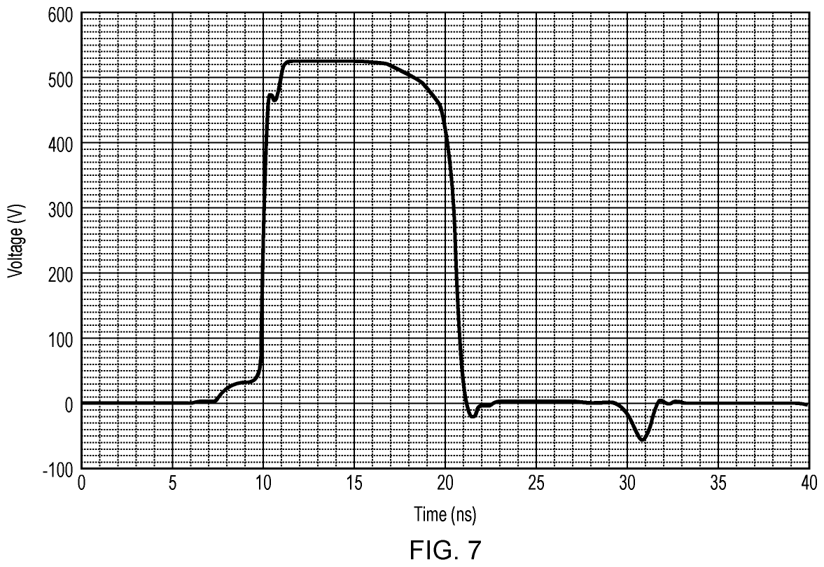

Taking this into account, the impedance of the load R_{L} was adjusted to 35Ω. This resulted in a single monopolar pulse at the load with zero reflection and no secondary pulse, as shown in FIG. 7.

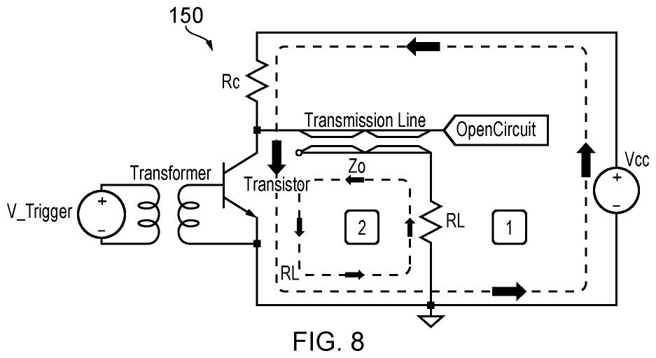

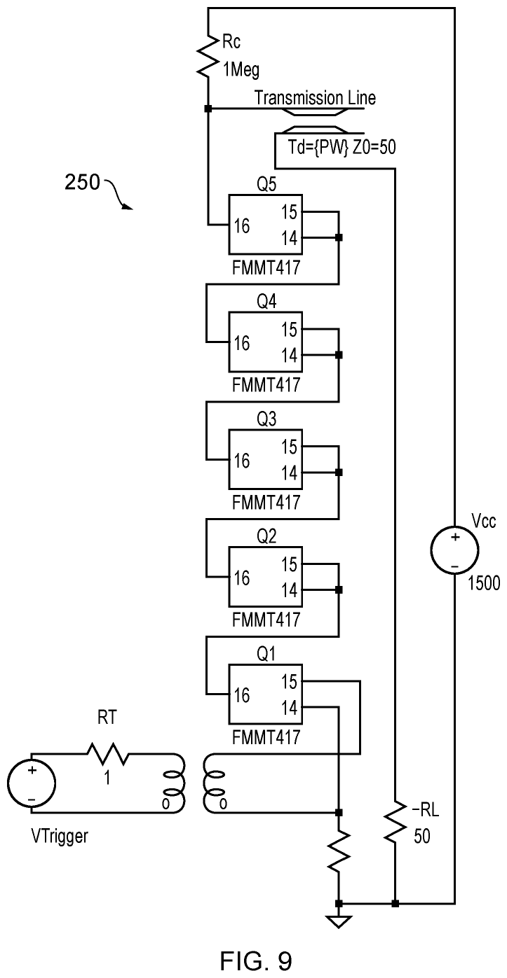

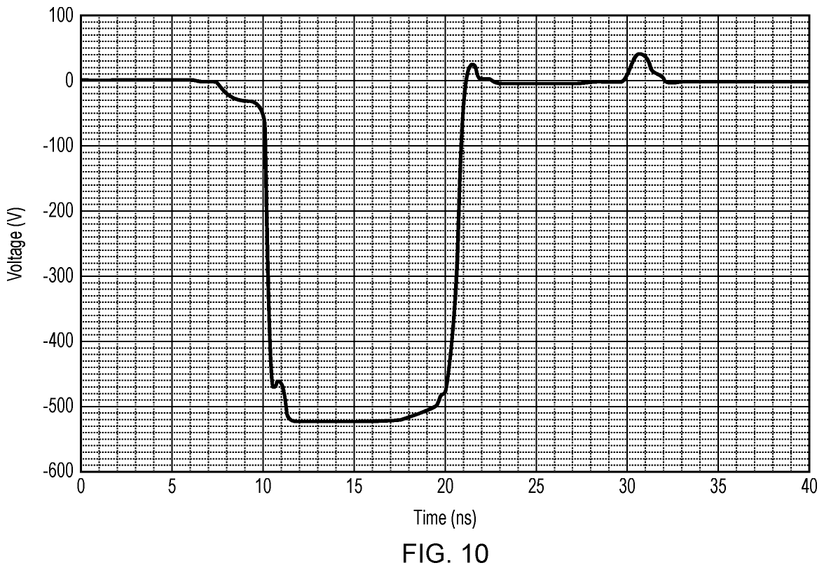

The circuit shown above in FIGS. 4 and 5 is configured to generate positive monopolar pulses. However, the circuit can also be adapted to generate a negative monopolar pulse by changing the position in which the load is connected. FIG. 8 is a schematic diagram of a pulse generating circuit 150 that utilises an open circuit transmission line technique in combination with an avalanche transistor as a fast switching element similar to the circuit 100 in FIG. 4. The circuit 150 of FIG. 8 differs from the circuit of FIG. 4 in that the load R_{L} is connected so that current flows in the opposite direction from FIG. 4 when the coaxial transmission line discharges. FIG. 9 shows a pulse generation circuit 250 that is an embodiment of the invention. The pulse generation circuit 250 is similar to the circuit shown in FIG. 5, except that the load R_{L} is connected so that current flows in the opposite direction from FIG. 5 when the coaxial transmission line discharges. FIG. 10 shows a graph of a negative monopolar pulse observed with a matched 35Ω load, obtained using the circuit 250 shown in FIG. 9.

In a development of the concepts discussed above, the pulse generating circuit can be configured as a bipolar pulse generating circuit. The operation of such a circuit can be identical to the monopolar designs in FIGS. 4 and 9.

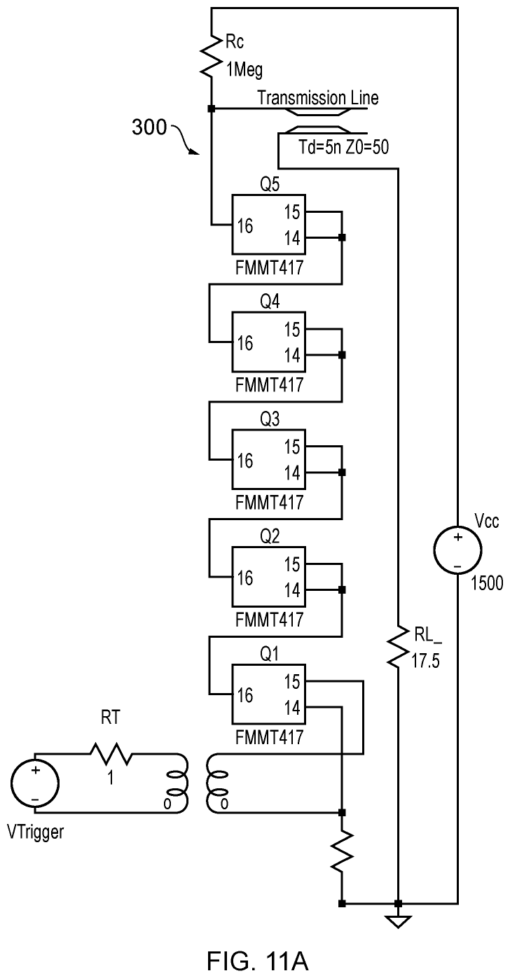

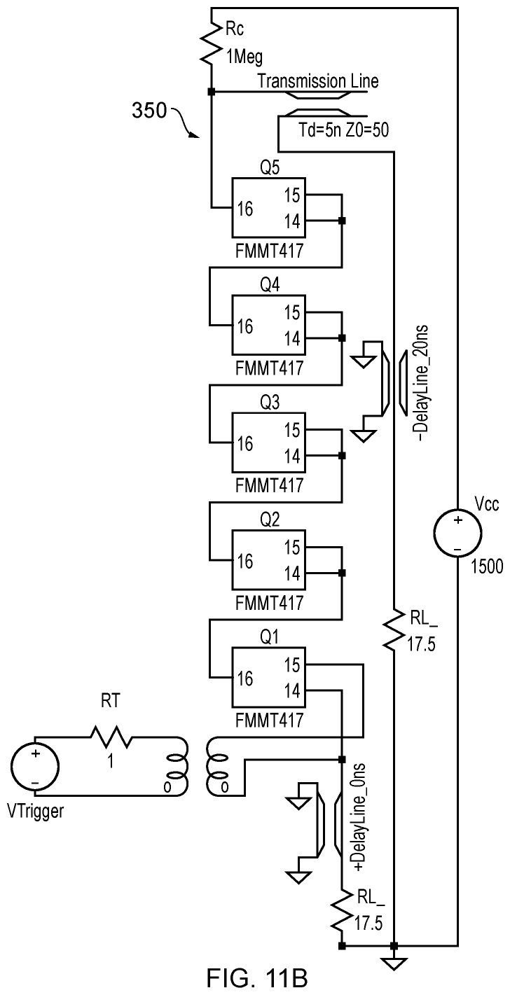

FIG. 11A is a schematic diagram of a pulse generation circuit 300 that is an embodiment of a bipolar pulse generating circuit 300. It is similar to the circuits shown in FIGS. 4 and 9, except that the pulse is generated on two separate loads, which are marked at R_{L}+ and R_{L}− in FIG. 11A.

These load location correspond to the locations for the positive and negative pulses discussed above.

The bipolar pulse generating circuit 300 produces a bipolar pulse, as the voltage difference across R_{L}; produces a positive pulse, where the voltage difference across R_{L}− produces a negative pulse. When the circuit 300 is used, these pulses observed at on R_{L}+ and R_{L}− simultaneously and are symmetrical, i.e. with the same pulse width, rise time, amplitude a repletion rate, but of different polarity.

As there are two loads in this circuit, the optimisation equations to reduce reflection must be revised. For a bipolar design the total load impedance, R_{ZL}=R_{L}+R_{L}−, is the impedance between the transmission line's outer conductor and the emitter of avalanche transistor Q1 and is the sum impedance of R_{L}+ and R_{L}−. The reflection coefficient can therefore be expressed using transmission line theory as:

$$Γ = ( \frac{( {R}_{Σ L} + {nR}_{A} ) - {Z}_{0}}{( {R}_{Σ L} + {nR}_{A} ) + {Z}_{0}} ) = ( \frac{( {R}_{L -} + {R}_{L +} + {nR}_{A} ) - {Z}_{0}}{( {R}_{L -} + {R}_{L +} + {nR}_{A} ) + {Z}_{0}} ) \\ ∴ if {R}_{Σ L} = {R}_{L -} + {R}_{L +} = {Z}_{0} - {nR}_{A} , Γ = 0$$

Similarly, the peak-to-peak voltage V_{ZL} over the loads can be expressed using DC theory as:

$${V}_{Σ L} = {V}_{L +} + {V}_{L -} = ( \frac{{R}_{Σ L}}{{Z}_{0} + {R}_{A} + {R}_{Σ L}} ) {V}_{cc} \\ {V}_{L -} = ( \frac{{R}_{L -}}{{Z}_{0} + {R}_{A} + {R}_{Σ L}} ) {V}_{cc} \\ {V}_{L +} = ( \frac{{R}_{L +}}{{Z}_{0} + {R}_{A} + {R}_{Σ L}} ) {V}_{cc}$$

wherein V_{L}+ and V_{L}− are the amplitudes of the positive and negative pulses respectively.

From the above, R_{L}+ and R_{L}− values of 17.5Ω would produce a bipolar pulse of a single pulse with zero reflection (Γ=0), and a simultaneous symmetrical pulse width of 2T and rise times<2 ns. Put another way, the bipolar pulse generating circuit 300 operates to create a single positive pulse of amplitude V_{EL} between the transmission line's outer conductor and the emitter of avalanche transistor Q1, across R_{EL} with a pulse width of 2T and zero reflection.

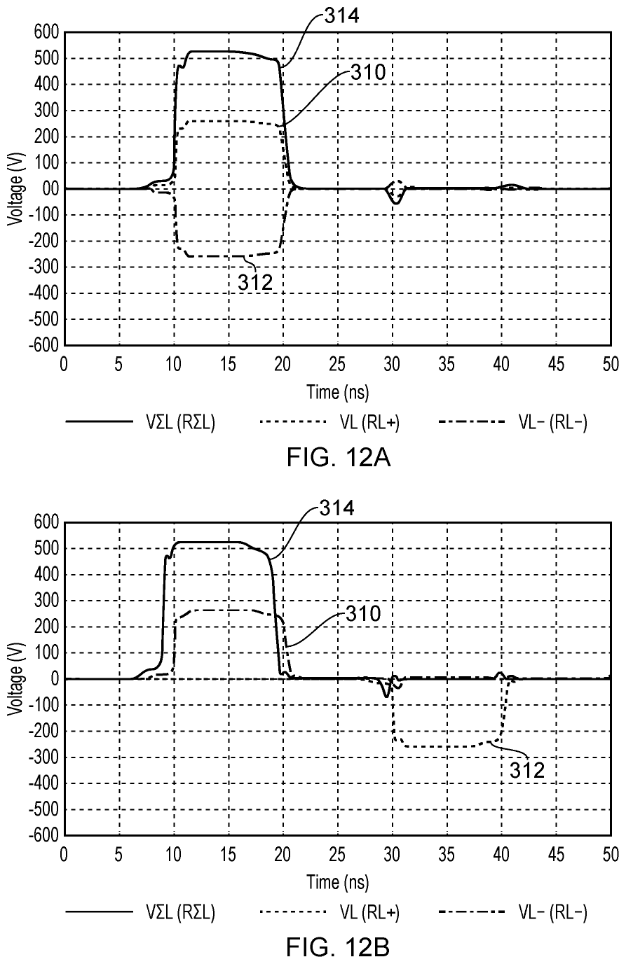

FIG. 12A is a graph that shows a pulse 310 observed at R_{L}+, a pulse 312 observed at R_{L}−, and a pulse 314 observed at R_{EL}. These observations verify the theory presented above. In FIG. 12A, a 5 ns transmission line produces a 10 ns pulse at all three loads with identical rise times (<2 ns). As R_{L}+=R−=17.5Ω, there is no reflection, i.e.

$$Γ - ( \frac{( {R}_{L -} + {R}_{L +} + {nR}_{A} ) - {Z}_{0}}{( {R}_{L -} + {R}_{L +} + {nR}_{A} ) + {Z}_{0}} ) - ( \frac{( 17.5 + 17.5 + 15 ) - 50 )}{( 17.5 + 17.5 + 15 ) - 50 )} ) - 0$$

The magnitude of V_{L}+, and V_{L}− is 262.5 V, so the peak-to-peak voltage V_{EL} is 520 V, which is the same as the equivalent monopolar design.

FIG. 11B is a schematic diagram of a bipolar pulse generation circuit 350 that is another embodiment of the invention. The circuit in FIG. 11B differs from FIG. 11A by providing a delay line before each of the loads (R_{L}+ and R_{L}−).

Placing a delay line before one or both loads allows manipulation of a delay between the two pulses. A delayed pulse will follow a non-delayed paired pulse by the delay time minus pulse width. In FIG. 11B, a 20 ns delay line is placed before R_{L}−.

FIG. 12B is a graph similar to FIG. 12A that shows a pulse 310 observed at R_{L}+, a pulse 312 observed at R_{L}−, and a pulse 314 observed at R_{EL}. FIG. 12B confirms the effect of the introducing the delay line, as all the three pulses in FIG. 12B and there parameters are identical to the FIG. 12A. The only difference is that the negative pulse across R_{L}− follows the positive pulse by 10 ns (i.e. 20 ns−10 ns).

The bipolar pulse generation circuit configuration discussed herein is thus capable of producing:
a symmetrical bipolar pulse, with positive and negative parts generated simultaneously or sequentially (i.e. with differing delays)
zero reflection but adjustable V_{L}+ and V_{L}− amplitudes, because the amplitudes are controlled by the ratio of R_{L}+ and R_{L}− but the reflection will remain zero if R_{EL}=R_{L}−+R_{L}+=Z₀−nR_{A}, condition is met.

In a further development, one or both of the delay lines may have an adjustable length that allows the introduced delay to be controlled. This may permit the separation of the positive and negative pulses to be adjusted on the fly, e.g. so that the instrument is capable of generating a variety of electroporation waveforms.

### REFERENCES

[1] W. Meiling and F. Stary, Nanosecond pulse techniques. New York: Gordon and Breach, 1970, p. 304.
[2] Q. Yang, X. Zhou, Q.-g. Wang and M. Zhao, “Comparative analysis on the fast rising edge pulse source with two kinds of avalanche transistor,” in Cross Strait Quad-Regional Radio Science and Wireless Technology Conference, Chengdu, 2013.
[3] G. Yong-sheng et al., “High-speed, high-voltage pulse generation using avalanche transistor,” Review of Scientific Instruments, vol. 87, no. 5, p. 054708, 2016.

1. A bipolar pulse generating circuit for an electrosurgical generator, the bipolar pulse generating circuit comprising:
a voltage source connectable to a load via a switching element;
a coaxial transmission line having an inner conductor separated from an outer conductor by a dielectric material, wherein the inner conductor has a first end connected between an input of the switching element and the voltage source and a second end in an open circuit condition, whereby the coaxial transmission line is charged by the voltage source when the switching element is in an OFF state and to be discharged when the switching element is in an ON state;
a first output connectable to the load, wherein the first output is located between an output of the switching element and ground to support a positive pulse when the coaxial transmission line discharges; and
a second output connectable to the load, wherein the second output is located between the outer conductor of the coaxial transmission line and ground to support a negative pulse when the coaxial transmission line discharges,
wherein the impedance of the coaxial transmission line is configured to match a sum of (i) the impedance the switching element, (ii) the impedance of the load at the first output, and (iii) the impedance of the load at the second output,
wherein a delay line is connected to either the first output or the second output, whereby supply of the positive pulse and negative pulse at the first output and second output occurs sequentially.

2. (canceled)

3. A bipolar pulse generating circuit according to claim 1, wherein the delay line has an adjustable length.

4. A bipolar pulse generating circuit according to claim 1, wherein the delay line is another length of coaxial transmission line.

5. A bipolar pulse generating circuit according to claim 1, wherein the switching element comprises:
a plurality of series connected avalanche transistors; and
a trigger pulse generator configured to generate a trigger pulse to activate the plurality of series connected avalanche transistors.

6. A bipolar pulse generating circuit according to claim 5, wherein the trigger pulse generator comprises a TTL device.

7. A bipolar pulse generating circuit according to claim 5, wherein the trigger pulse has a voltage less than the emitter-base breakdown voltage of each of the plurality of avalanche transistors.

8. A bipolar pulse generating circuit according to claim 5, wherein the trigger pulse generator is connected to the plurality of series connected avalanche transistors via a transformer.

9. A bipolar pulse generating circuit according to claim 5, wherein the trigger pulse is applied between the collector and emitter of a first transistor of the plurality of series connected avalanche transistors.

10. A bipolar pulse generating circuit according to claim 9, wherein the first transistor is furthest from the coaxial transmission line.

11. A bipolar pulse generating circuit according to claim 5, wherein a diode is connected in parallel with each of the plurality of series connected avalanche transistors to clamp the voltage across each transistor to less than its collector-base breakdown voltage.

12. A bipolar pulse generating circuit according to claim 5, wherein each transistor in the plurality of series connected avalanche transistors is identical.

13. A bipolar pulse generating circuit according to claim 1, wherein the coaxial transmission line has a length selected to provide a line delay equal to or less than 5 ns.

14. A bipolar pulse generating circuit according to claim 1, wherein the coaxial transmission line is charged by the voltage source through a resistor.

15. A bipolar pulse generating circuit according to claim 1, wherein the load is an electrosurgical instrument.

16. An electrosurgical generator having a bipolar pulse generating circuit according to claim 1.
